(19) United States
(12) Patent Application Publication
(10) Pub. No.: US 2009/0087862 A1
(43) Pub. Date: Apr. 2, 2009

(54) Chemical Sensing Device

(75) Inventors: Timothy Joseph Nicholas Carter, Sittingbourne (GB); Steven Andrew Ross, Sittingbourne (GB)

Correspondence Address: ROTHWELL, FIGG, ERNST & MANBECK, P.C., 1425 K STREET, N.W., SUITE 800, WASHINGTON, DC, 20005, US

(73) Assignee: VIVACTA LTD., Sittingbourne, Kent (GB)

(21) Appl. No.: 12/293,141

(22) PCT Filed: Mar. 16, 2007
(86) PCT No.: PCT/GB2007/000938

§ 371 Date: Dec. 17, 2008

Related U.S. Application Data
(60) Provisional application No. 60/786,808, filed on Mar. 29, 2006.
(60) Provisional application No. 60/811,110, filed on Jun. 6, 2006.

(30) Foreign Application Priority Data
Mar. 17, 2006 (GB) 0605452.2

(51) Int. Cl. G01N 33/53

(52) U.S. Cl. 435/7.1

(57) ABSTRACT

The present invention relates to chemical sensing and in particular to a method for detecting an analyte in a sample of whole blood. The method comprise, in summary, the steps of: exposing the sample to a transducer having a tethered reagent; introducing a labelled reagent; irradiating the sample with a series of pulses of electromagnetic radiation at a wavelength of 600 run or above; and transducing and detecting the electrical signal and the time delay between each pulse. The label on the labelled reagent absorbs the electromagnetic radiation at a level which is at least equal to the absorption of the sample of whole blood at the wavelength of the electromagnetic radiation used.

The present invention relates to a chemical sensing device and in particular to a chemical sensing device employing a transducer.

The monitoring of analytes in solution, such as biologically important compounds in bioassays, has a broad applicability. Accordingly, a wide variety of analytical and diagnostic devices are available. Many devices employ a reagent which undergoes an eye-detectable colour change in the presence of the species being detected. The reagent is often carried on a test strip and optics may be provided to assist in the measurement of the colour change.

WO 90/13017 discloses a pyroelectric or other thermoelectric transducer element in a strip form. Thin film electrodes are provided and one or more reagents are deposited on the transducer surface. The reagent undergoes a selective calorimetric change when it comes into contact with the species being detected. The device is then typically inserted into a detector where the transducer is illuminated usually from below by an LED light source and light absorption by the reagent is detected as microscopic heating at the transducer surface. The electrical signal output from the transducer is processed to derive the concentration of the species being detected.

The system of WO 90/13017 provides for the analysis of species which produce a colour change in the reagent on reaction or combination with the reagent. For example, reagents include pH and heavy metal indicator dyes, reagents (e.g. o-cresol in ammoniacal copper solution) for detecting aminophenol in a paracetamol assay, and a tetrazolium dye for detecting an oxidoreductase enzyme in an enzyme-linked immuno-sorbant assay (ELISA). However, while this system is useful in certain applications, it has been considered suitable only for analysis where the species being analysed generates a colour change in the reagent since it is the reagent which is located on the surface of the transducer. Therefore, this system cannot be applied to the analysis of species which do not cause a colour change in the reagent or when the colour change is not on the surface of the transducer. In the field of bioassays, this gives the system limited applicability.

WO 2004/090512 discloses a device based on the technology disclosed in WO 90/13017, but relies on the finding that energy generated by non-radiative decay in a substance on irradiation with electromagnetic radiation may be detected by a transducer even when the substance is not in contact with the transducer, and that the time delay between the irradiation with electromagnetic radiation and the electrical signal produced by the transducer is a function of the distance of the substance from the surface of the film. This finding provided a device capable of “depth profiling” which allows the device to distinguish between an analyte bound to the surface of the transducer and an analyte in the bulk liquid. This application therefore discloses a device which is able to be used in assays, typically bioassays, without having to carry out a separate washing step between carrying out a binding event and detecting the results of that event.

The device disclosed in WO 2004/090512 has found wide applicability but the applicability is limited when the device is used to detect the presence of an analyte in whole blood (uncoagulated blood containing cells). This is because a random distribution of red cells in the sample implies that a fraction of those cells will be close enough to the piezofilm to generate a background signal. The very high absorption properties of red cells means that this signal can interfere with the binding assay being performed and limit its applicability and sensitivity.

There remains a requirement in the art, therefore, for a system which can operate in the presence of a sample of whole blood. This is particularly important since many assays are performed on analytes present in blood.

Accordingly, the present invention provides a method for detecting an analyte in a sample of whole blood, comprising the steps of: exposing the sample to a transducer having a pyroelectric or piezoelectric element and electrodes which is capable of transducing a change in energy to an electrical signal, the transducer having at least one tethered reagent on or proximal thereto, the at least one tethered reagent having a binding site which is capable of binding the analyte; introducing a labelled reagent into the sample, wherein the labelled reagent contains a binding site for the analyte or the tethered reagent and a label which is capable of absorbing electromagnetic radiation generated by a radiation source to generate energy by non-radiative decay; irradiating the sample with a series of pulses of electromagnetic radiation at a wavelength of 600 nm or above, transducing the energy generated into an electrical signal; detecting the electrical signal and the time delay between each pulse of electromagnetic radiation from the radiation source and the generation of the electric signal, wherein the time delay between each of the pulses of electromagnetic radiation and the generation of the electric signal corresponds to the position of the labelled reagent at any of one or more positions at different distances from the surface of the transducer, wherein the label on the labelled reagent is selected such that the label absorbs the electromagnetic radiation at a level which is at least equal to the absorption of the sample of whole blood at the wavelength of the electromagnetic radiation used.

The present invention also provides a kit comprising (i) a device for detecting an analyte in a liquid sample containing suspended particles comprising a radiation source adapted to generate a series of pulses of electromagnetic radiation at a wavelength of 600 nm or above, a transducer having a pyroelectric or piezoelectric element and electrodes which is capable of transducing a change in energy to an electrical signal, at least one tethered reagent on or proximal to the transducer, the tethered reagent having a binding site which is capable of binding the analyte, and a confinement structure for holding the sample in fluid contact with transducer, a detector which is capable of detecting the electrical signal generated by the transducer and which is adapted to determine the time delay between each pulse of electromagnetic radiation from the radiation source and the generation of the electric signal; and (ii) a labelled reagent which has a binding site which binds to the analyte or the tethered reagent and a label which is capable of absorbing electromagnetic radiation generated by a radiation source to generate energy by non-radiative decay, wherein the label on the labelled reagent is selected such that the label absorbs the electromagnetic radiation at a level which is at least equal to the absorption of the sample of whole blood at the wavelength of the electromagnetic radiation used.

This method/kit allows the user to detect the presence of an analyte in a sample of whole blood by combining the technique of depth profiling with a careful selection of the label being detected.

The present invention will now be described with reference to the drawings, in which

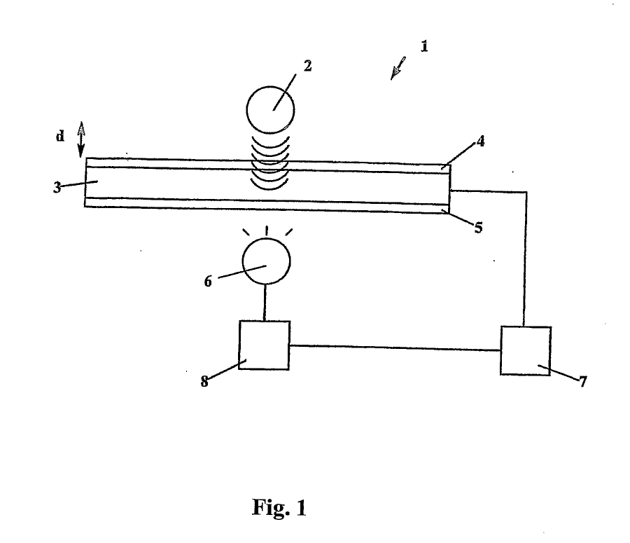

FIG. 1 shows a schematic representation of the chemical sensing device of the present invention;

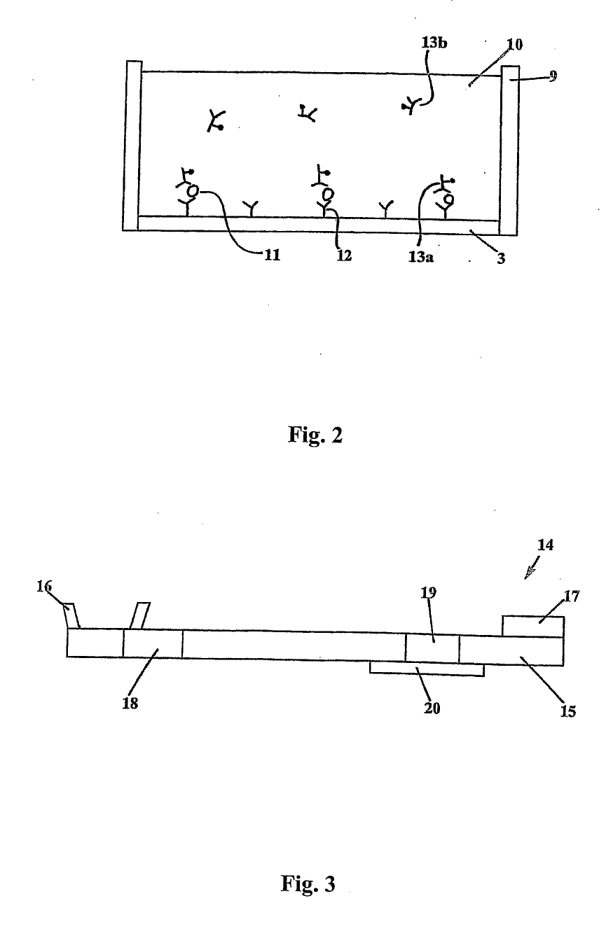

FIG. 2 shows a sandwich immunoassay using the device of the present invention;

FIG. 3 shows a lateral-flow assay device in accordance with the present invention;

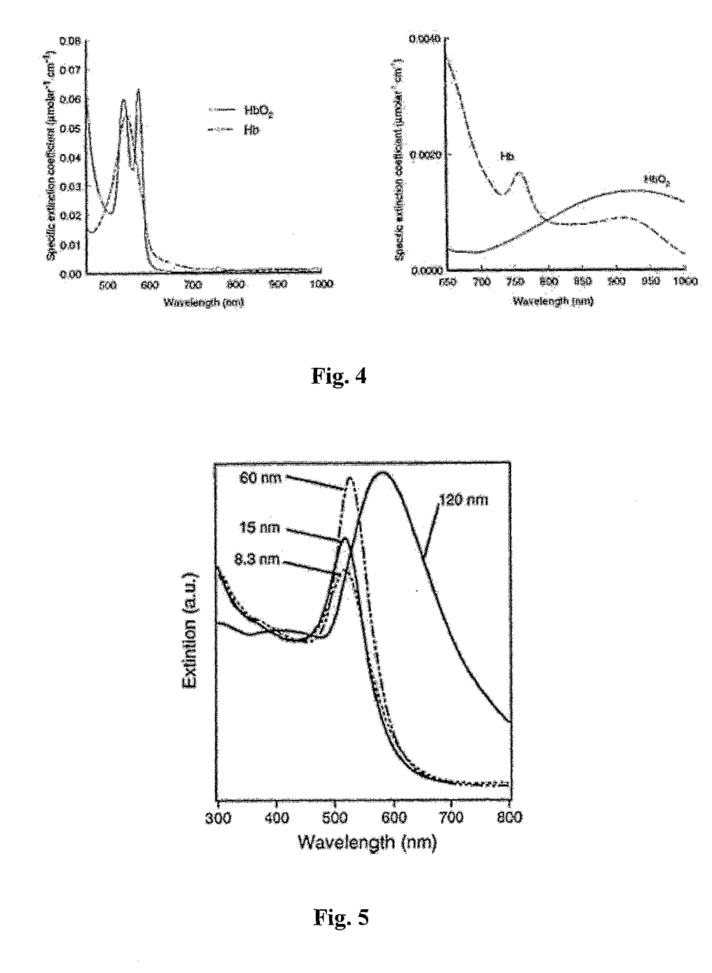

FIG. 4 shows the absorption profile of haemoglobin and oxygenated haemoglobin;

FIG. 5 shows the absorption profile of gold particles having different particle sizes;

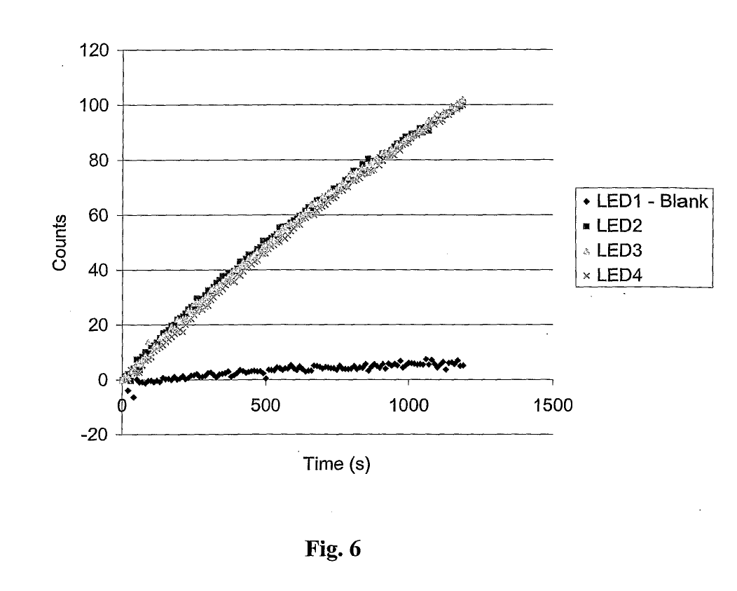

FIG. 6 shows the time course of the binding assay in Example 1; and

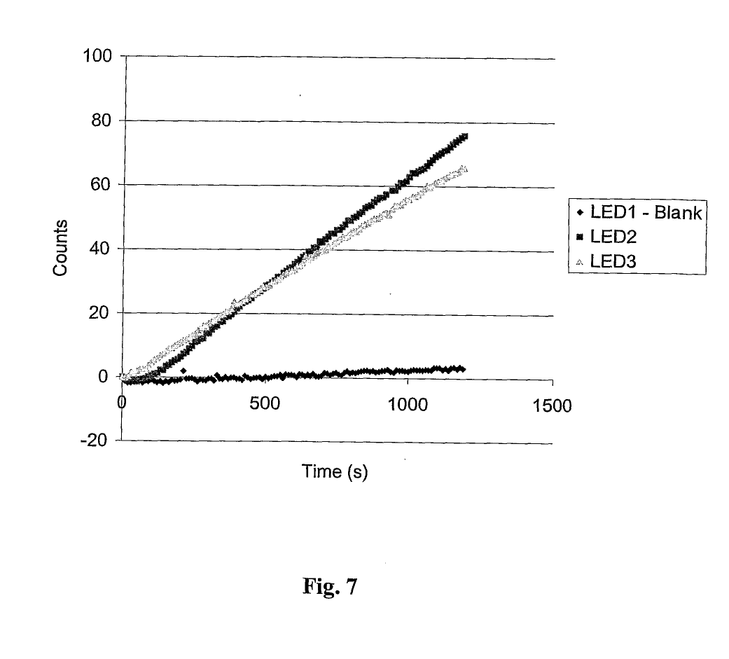

FIG. 7 shows the time course of the binding assay in Example 2.

FIG. 1 shows a chemical sensing device 1 of the type used with the present invention. The device 1 relies on heat generation in a substance 2 on irradiation of the substance 2 with electromagnetic radiation. The device 1 comprises a pyroelectric or piezoelectric transducer 3 having electrode coatings 4,5. The transducer 3 is preferably a poled polyvinylidene fluoride film. The electrode coatings 4,5 are preferably formed from indium tin oxide having a thickness of about 35 nm, although almost any thickness is possible from a lower limit of 1 nm below which the electrical conductivity is too low and an upper limit of 100 nm above which the optical transmission is too low (it should not be less than 95% T). A substance 2 is held proximal to the transducer 3 using any suitable technique, shown here attached to the upper electrode coating 4. The substance may be in any suitable form and a plurality of substances may be deposited. Preferably, the substance 2 is adsorbed on to the upper electrode, e.g. covalently coupled or bound via intermolecular forces such as ionic bonds, hydrogen bonding or van der Waal's forces. A key feature of this device is that the substance 2 generates heat when irradiated by a source of electromagnetic radiation 6, such as light, preferably visible light. The light source may be, for example, an LED. The light source 6 illuminates the substance 2 with light of the appropriate wavelength (e.g. a complementary colour). Although not wishing to be bound by theory, it is believed that the substance 2 absorbs the light to generate an excited state which then undergoes non-radiative decay thereby generating energy, indicated by the curved lines in FIG. 1. This energy is primarily in the form of heat (i.e. thermal motion in the environment) although other forms of energy, e.g. a shock wave, may also be generated. The energy is, however, detected by the transducer and converted into an electrical signal. The device of the present invention is calibrated for the particular substance being measured and hence the precise form of the energy generated by the non-radiative decay does not need to be determined. Unless otherwise specified the term “heat” is used herein to mean the energy generated by non-radiative decay. The light source 6 is positioned so as to illuminate the substance 2. Preferably, the light source 6 is positioned below the transducer 3 and electrodes 4,5 and the substance 2 is illuminated through the transducer 3 and electrodes 4,5. The light source may be an internal light source within the transducer in which the light source is a guided wave system. The wave guide may be the transducer itself or the wave guide may be an additional layer attached to the transducer.

The energy generated by the substance 2 is detected by the transducer 3 and converted into an electrical signal. The electrical signal is detected by a detector 7. The light source 6 and the detector 7 are both under the control of the controller 8. The light source 6 generates a series of pulses of light (the term “light” used herein means any form of electromagnetic radiation unless a specific wavelength is mentioned) which is termed “chopped light”. In principle, a single flash of light, i.e. one pulse of electromagnetic radiation, would suffice to generate a signal from the transducer 3. However, in order to obtain a reproducible signal, a plurality of flashes of light are used which in practice requires chopped light. The frequency at which the pulses of electromagnetic radiation are applied may be varied. At the lower limit, the time delay between the pulses must be sufficient for the time delay between each pulse and the generation of an electrical signal to be determined. At the upper limit, the time delay between each pulse must not be so large that the period taken to record the data becomes unreasonably extended. Preferably, the frequency of the pulses is from 2-50 Hz, more preferably 5-15 Hz and most preferably 10 Hz. This corresponds to a time delay between pulses of 20-500 ms, 66-200 ms and 100 ms, respectively. However, the time delay may be as low as 1 ms. In addition, the so-called “mark-space” ratio, i.e. the ratio of on signal to off signal is preferably one although other ratios may be used without deleterious effect. Sources of electromagnetic radiation which produce chopped light with different frequencies of chopping or different mark-space ratios are known in the art. The detector 7 determines the time delay (or “correlation delay”) between each pulse of light from light source 6 and the corresponding electrical signal detected by detector 7 from transducer 3. The applicant has found that this time delay is a function of the distance, d.

Any method for determining the time delay between each pulse of light and the corresponding electrical signal which provides reproducible results may be used. Preferably, the time delay is measured from the start of each pulse of light to the point at which a maximum in the electrical signal corresponding to the absorption of heat is detected as by detector 7.

Thus substance 2 may be separated from the transducer surface and a signal may still be detected. Moreover, not only is the signal detectable through an intervening medium capable of transmitting energy to the transducer 3, but different distances, d, may be distinguished (this has been termed “depth profiling”) and that the intensity of the signal received is proportional to the concentration of the substance 2 at the particular distance, d, from the surface of the transducer 3.

In a typical immunoassay, an antibody specific for an antigen of interest is attached to a polymeric support such as a sheet of nitrocellulose, polyvinylchloride or polystyrene. A drop of a sample is laid on the sheet, which is washed after formation of the antibody-antigen complex. Antibody specific for a different site on the antigen is then added, and the sheet is again washed. This second antibody carries a label so that it can be detected with high sensitivity. The amount of second antibody bound to the sheet is proportional to the quantity of antigen in the sample. This assay and other variations on this type of assay are well known, see, for example, “The Immunoassay Handbook, 2nd Ed.” David Wild, Ed., Nature Publishing Group, 2001. The device of the present invention may be used in any of these assays.

FIG. 2 shows a typical capture antibody assay using a piezoelectric or pyroelectric transducer. A device includes a transducer 3 and a well 9 holding a liquid 10 containing an analyte 11 dissolved or suspended therein. The well 9 acts as a confinement structure holding the sample in fluid contact with the transducer 3. The transducer 3 has a number of tethered reagents attached thereto, i.e. antibody 12. The antibody 12 is shown attached to the film in FIG. 2 and this attachment may be via a covalent bond or by non-covalent adsorption onto the surface, such as by hydrogen bonding. Although the antibody is shown as attached to the transducer, any technique for holding the antibody 12 on or proximal to the transducer 3 is applicable. For example, an additional layer may separate the antibody 12 and the transducer 3, such as a silicone polymer layer, or the antibody could be attached to inert particles and the inert particles are then attached to the transducer 3. Alternatively, the antibody 12 could be entrapped within a gel layer which is coated onto the surface of the transducer 3.

In use, the well is filled with liquid 10 (or any fluid) containing an antigen 11. The antigen 11 then binds to antibody 12. Additional labelled antibody 13 is added to the liquid and a so-called “sandwich” complex is formed between the bound antibody 12, the antigen 11 and the labelled antibody 13. An excess of labelled antibody 13 is added so that all of the bound antigen 11 forms a sandwich complex. The sample therefore contains bound labelled antigen 13a and unbound labelled antigen 13b free in solution.

During or following formation of the sandwich complex, the sample is irradiated using a series of pulses of electromagnetic radiation, such as light. The time delay between each pulse and the generation of an electrical signal by the transducer 3 is detected by a detector. The appropriate time delay is selected to measure only the heat generated by the bound labelled antigen 13a. Since the time delay is a function of the distance of the label from the transducer 3, the bound labelled antibody 13a may be distinguished from the unbound labelled antigen 13b. This provides a significant advantage over the conventional sandwich immunoassay in that it removes the need for washing steps. In a conventional sandwich immunoassay, the unbound labelled antibody must be separated from the bound labelled antibody before any measurement is taken since the unbound labelled antigen interferes with the signal generated by the bound labelled antigen. However, on account of the “depth profiling” provided by the present invention, bound and unbound labelled antigen may be distinguished.

The tethered reagent is attached to the transducer 3 and hence is distinct from the labelled reagent which is not tethered to the transducer and is free to diffuse through the liquid.

As a further example of known immunoassays, the present invention may be applied to competitive assays in which the electrical signal detected by the detector is inversely proportional to the presence of an unlabelled antigen in the sample. In this case, it is the amount of the unlabelled antigen in the sample which is of interest.

In a competitive immunoassay, an antibody is attached to the transducer as shown in FIG. 2. A sample containing the antigen is then added. However, rather than adding a labelled antibody, a known amount of labelled antigen is added to the solution. The labelled and unlabelled antigens then compete for binding to the antibodies attached to the transducer 3. The concentration of the bound labelled antigen is then inversely proportional to the concentration of bound unlabelled antigen and hence, since the amount of labelled antigen is known, the amount of unlabelled antigen in the initial solution may be calculated. The same labels specified with reference to the antibodies may also be used with the antigens. In this embodiment, the labelled reagent is therefore a labelled analyte. Many forms of competitive immunoassay are known in the art include those where the antibody in solution is labelled and an antigen is bound to the sensor surface (see, for example, Wild, the Immunoassay Handbook given above).

In another embodiment, the piezoelectric or pyroelectric transducer is applied to lateral-flow analysis. This has particular application for the detection of human chorionic gonadotrophin (HCG) in pregnancy testing.

FIG. 3 shows a simplified lateral flow device 14 in accordance with the present invention. The device has a filter paper or other absorber 15 containing a sample receiver 16 and a wick 17 together with first and second zones 18 and 19 containing unbound and bound antibodies (i.e. unbound and bound to the filter paper or other absorber 15), respectively, capable of binding to HCG. The first and second zones 18 and 19 are made of a porous material which confines the liquid sample as the sample passes through the zone. The device also contains a piezoelectric film 20 proximal to the second zone 19. A sample of urine or serum is added to the sample receiver 16 which then travels along the absorber 15 to the wick 17. The first zone 18 contains a labelled antibody to HCG and as the sample passes through the first zone 18, if HCG is present in the sample, the labelled antibody to HCG is picked up by the sample. As the sample passes from the first zone 18 to the second zone 19, the antigen and antibody form a complex. At the second zone 19, a second antibody is attached either to the absorber 15 or the piezoelectric film 20 which is capable of binding the antigen-antibody complex. In a conventional lateral-flow analysis such as a pregnancy tester, a positive result produces a colour change at the second zone 19. However, the conventional lateral-flow analysis is restricted to clear samples and is essentially suitable only for a positive or negative i.e. yes/no, result. The device of the present invention, however, uses a piezoelectric film 20. Since only the sample at the predetermined distance from the film is measured, contaminants in the bulk sample will not affect the reading. Moreover, the sensitivity of the piezoelectric film provides a quantification of the result. Quantification of the result provides a broader applicability to the lateral-flow analysis and also distinguishing between different quantities of antigens reduces the number of erroneous results.

In assays of the types described above it would be expected that since only the signal at a known distance from the transducer 3 is determined, any other components of the sample which are free in solution or suspension should not interfere with the detection. However, it has been found that whole blood may interfere with the reading, as described previously, and lower sensitivity. Typically in the art this problem is addressed by carrying out an initial separation of the sample to remove red blood cells and other contaminants from a sample of whole blood. However, the present invention avoids the need for a separation step when using a sample of whole blood by using a label which absorbs at a different wavelength to the sample.

On account of the high molar absorption coefficient of gold particles, assays of this type typically use these particles and usually 40 nm particles. The 40 nm gold particles are used in the art because this size of particle moves readily by capillary action through the filter membranes which make up the solid phase of lateral flow devices, and because larger particles are slow to resuspend. However, 40 nm gold particles also have their absorption maximum at around 525 nm and hence red blood cells present in a whole blood sample interfere with the assay. For this reason, conventional assays carried out on whole blood incorporate a separation step to separate the red blood cells from the whole sample. The present applicant has found, however, that by using a device incorporating the depth profiling methodology described herein, larger gold particles may be used which have a different absorption profile.

FIG. 4 shows the absorption spectra for haemoglobin (Hb) and oxygenated haemoglobin (HbO₂). The molar extinction coefficient at 525 nm is around 30000. The molar extinction coefficients at 654 mm are approximately 345 for HbO₂ and 3500 for Hb. The concentration of haemoglobin in blood is around 0.0023 mol dm⁻³, giving absorption values (optical densities) of approximately 70 at 525 nm, 8 for venous blood at 654 (i.e. non-oxygenated blood) and 0.8 for arterial blood (i.e. oxygenated blood). This intense optical density of 70 at 525 nm would be expected to generate a significant signal on the piezofilm due to the fraction of red cells in the sample which are in close proximity to the film.

FIG. 5 shows the absorbance maxima for different sizes of gold particles (note that the scale is arbitrary, the larger particles will have much higher molar extinction coefficients). It is clear that there is a great deal of overlap between the absorption maxima of haemoglobin and the absorption maxima of the smaller gold particles of up to 60 nm particle size.

Thus, by using larger gold particles than those presently used in the art, an assay may be carried out in the presence of whole blood. Using larger gold particles also have the advantage that the molar extinction coefficient is greater. The molar extinction coefficient increases roughly with the cube of the particle diameter. This means that an 80 nm will exhibit an approximately eight-fold increase in absorptivity over that of a 40 nm particle providing an increase in the sensitivity of the assay.

Preferably, the present invention uses a gold particle label having a particle size of 50-250 mm, more preferably minimum size of 80 nm or more, most preferably 100 nm or more, and a maximum size of 200 nm or less and most preferably 150 nm or less. By particle size is meant the diameter of the particle at its widest point. Gold particles are commercially available or may be prepared using known methods (see for example G. Frens, Nature, 241, 20-22 (1973)).

The label is preferably selected from a dye molecule, a gold particle, a coloured polymer particle (e.g. a coloured latex particle), a fluorescent molecule, an enzyme, a magnetic particle and a carbon particle. However, any label capable of interacting with electromagnetic radiation to generate heat would be acceptable, providing it absorbs at the appropriate frequency. In the case of a magnetic particle, the electromagnetic radiation is radio frequency radiation. All of the other labels mentioned hereinabove employ light. In the case of a gold particle, to increase the signal further, the label may be enhanced using a solution of silver ions and a reducing agent. The gold catalyses/activates the reduction of the silver ions to silver metal and it is the silver metal which absorbs the light.

The label may also be a nanoparticle comprising a non-conducting core material and at least one metal shell layer. The metal of the metal shell layer may be selected from coinage metals, noble metals, transition metals, and synthetic metals, but is preferably gold. The core material is preferably a dielectric material or semiconductor. Suitable dielectric materials include but are not limited to silicon dioxide, titanium dioxide, polymethyl methacrylate (PMMA), polystyrene, gold sulfide and macromolecules such as dendrimers. Monodisperse colloidal silica is particularly preferred as this material readily forms spherical particles. Gold-plated silica is a particularly preferred label. For any given particle, the maximum absorbance depends upon the ratio of the thickness of the nonconducting layer to the conducting shell layer and these parameters may be varied to give the desired absorbance profile. Such labels are described in U.S. Pat. No. 6,344,272.

The electromagnetic radiation used in the present invention has a wavelength of 600 nm or above, preferably 610 nm or above. The upper limit is less important but is preferably less than 1000 nm, more preferably less than 800 nm. The wavelength is most preferably 654 nm. The source is preferably an LED.

The label absorbs the electromagnetic radiation at a level which is at least equal to the absorption of the sample of whole blood at the wavelength of the electromagnetic radiation used. The absorption is as measured using the concentrations used in the assay and prior to any binding event. Preferably, the absorption of the label is at least double that of the background and most preferably at least ten times the absorption.

The labelled antibody, or indeed any one or more additional reagents may be stored in a chamber incorporated into the device or may be supplied separate from the device in the form of a kit of parts.

An apparatus for measuring analyte levels in a blood sample preferably comprises a hand-held portable reader and a disposable device containing the piezoelectric or pyroelectric film. A small sample of blood (about 10 microlitres) is obtained and transferred to a chamber within the disposable device. One side of the chamber is made from the piezoelectric or pyroelectric film coated with an antibody capable of binding to the analyte of interest. An additional solution may then be added containing, for example, labelled antibody or a known concentration of labelled antigen as described above. The reaction is allowed to proceed and the disposable device is then inserted into the reader which activates the measurement process. The results of the assay are then indicated on a display on the reader. The disposable device containing the piezoelectric film is then removed and discarded.

The device described herein is not restricted to detecting only one analyte in solution. Since the device provides “depth profiling” different analytes may be detected by employing reagents which selectively bind each analyte being detected wherein the reagents are different distances from the surface of the transducer 3. For example, two analytes may be detected using two reagents, the first reagent being positioned at a first distance from the film and the second reagent being positioned at a second distance from the film. The time delay between each pulse of electromagnetic radiation and the generation of electrical signal will be different for the two analytes bound to the first and second reagents.

As well as providing different depths, multiple tests may be carried out using different types of reagents, e.g. different antibodies, at different parts of the transducer, i.e. specific areas or “spots” on the transducer surface. Alternatively, or in addition, multiple tests may be carried out using reagents/analytes which respond to different wavelengths of electromagnetic radiation.

The substance generating the heat may be on the surface of the film, however, the substance may be at least 5 nm from the surface of the film and, may be no more than 500 μm from the surface of the film. By selecting a suitable time delay, however, a substance in the bulk solution may also be measured.

As alternatives to antibody-antigen reactions, the reagent and analyte may be a first and second nucleic acid where the first and second nucleic acids are complementary, or a reagent containing avidin or derivatives thereof and an analyte containing biotin or derivatives thereof, or vice versa.

### EXAMPLES

A poled polyvinylidene fluoride bimorph, coated in indium tin oxide, is used as the sensing device in the following examples.

The sensing device is dip-coated in nitrocellulose solution to give a nitrocellulose layer of around 1 micron thickness on top of the indium tin oxide. This film is then constructed into a reaction chamber of 100 μL through the addition of a 500 μm layer of pressure sensitive adhesive and a polycarbonate lidding material. Holes are available for the addition and removal of liquid from the reaction chamber.

### Example 1

### Comparative Example

An experiment is carried out on the surface of a nitrocellulose-coated piezoelectric film to detect the presence of antibody-labelled particles in a solution adjacent to the film. Liquid is constrained on the nitrocellulose surface during the experiment. The film is submerged overnight in a solution of polymerised streptavidin at a concentration of 20 μg/ml in PBS (phosphate buffered saline) pH 7.2. Following a rinse/wash step with PBS/Tween 0.05%, biotinylated mouse antibody is added and allowed to incubate for one hour. After rinsing away excess mouse antibody, the surface is stabilised using a proprietary stabiliser.

A solution of goat anti-mouse antibody conjugated to 40 nm gold particles is diluted until the concentration of gold particles is 0.15 pmoles/ml. This solution is added to the stabilised mouse antibody coated film.

The film is then irradiated with chopped light of wavelength 525 nm (green light). The magnitude of the maximum signal detected by the piezoelectric film is measured. The signal is displayed using an analogue-to-digital converter. The signal received by the detector increases with time, as the binding of the gold particles to the surface takes place. The kinetic profile of the antibody-antigen reaction is monitored, with measurements being taken every 10 seconds over a period of 20 minutes.

A blank experiment is performed on this same film by substituting PBS for the biotinylated mouse antibody. The signal received by the detector does not increase with time for the blank experiment.

### Example 2

The surface of a nitrocellulose-coated PVDF film is coated in the same manner as in Example 1.

A solution of anti-mouse antibody conjugated to 80 nm gold particles is diluted until the concentration of gold particles in the solution is 0.015 pmoles/ml. This solution is added to the stabilised mouse antibody coated film.

The film is then irradiated with chopped light of wavelength 654 nm (red light). The magnitude of the maximum signal detected by the piezoelectric film is measured. The signal is displayed using an analogue-to-digital converter. The signal received by the detector increases with time. The kinetic profile of the antibody-antigen reaction is monitored over time with measurements being taken every 10 seconds over a period of 20 minutes.

A blank experiment is performed by substituting PBS for the biotinylated mouse antibody. The signal received by the detector does not increase with time for the blank experiment.

### Example 3

Example 3 is a repeat of Example 2 in the presence of whole blood and a similar signal is generated.

1. A method for detecting an analyte in a sample of whole blood, comprising the steps of:
exposing the sample to a transducer having a pyroelectric or piezoelectric element and electrodes which is capable of transducing a change in energy to an electrical signal, the transducer having at least one tethered reagent on or proximal thereto, the at least one tethered reagent having a binding site which is capable of binding the analyte;
introducing a labelled reagent into the sample, wherein the labelled reagent contains a binding site for the analyte or the tethered reagent and a label which is capable of absorbing electromagnetic radiation generated by a radiation source to generate energy by non-radiative decay;
irradiating the sample with a series of pulses of electromagnetic radiation at a wavelength of 600 nm or above,
transducing the energy generated into an electrical signal;
detecting the electrical signal and the time delay between each pulse of electromagnetic radiation from the radiation source and the generation of the electric signal, wherein the time delay between each of the pulses of electromagnetic radiation and the generation of the electric signal corresponds to the position of the labelled reagent at any of one or more positions at different distances from the surface of the transducer,
wherein the label on the labelled reagent is selected such that the label absorbs the electromagnetic radiation at a level which is at least equal to the absorption of the sample of whole blood at the wavelength of the electromagnetic radiation used.

2. A method as claimed in claim 1, wherein the at least one tethered reagent is an antibody and the analyte is an antigen.

3. A method as claimed in claim 1, wherein the labelled reagent is a labelled antibody.

4. A method as claimed in claim 1, wherein the at least one tethered reagent is an antibody, the analyte is an antigen and the labelled reagent is a labelled antigen which is also capable of binding to the at least one tethered reagent and the electrical signal detected by the detector is inversely proportional to the presence of the analyte in the sample.

5. A method as claimed in claim 1, wherein the at least one tethered reagent is a first nucleic acid and the analyte is a second nucleic acid and the first and second nucleic acids are complementary.

6. A method as claimed in claim 1, wherein the at least one tethered reagent contains avidin or derivatives thereof and the analyte contains biotin or derivatives thereof, or vice versa.

7. A method as claimed in claim 1, wherein the label on the labelled reagent is selected from a dye molecule, a gold particle, a coloured-polymer particle, a fluorescent molecule, an enzyme, a magnetic particle, a carbon particle and a nanoparticle comprising a non-conducting core material and at least one metal shell layer.

8. A method as claimed in claim 1, wherein the time delay is at least 1 millisecond.

9. A method as claimed in claim 1, wherein the time delay is no greater than 500 milliseconds.

10. A method as claimed in claim 1, wherein the electromagnetic radiation is light, preferably visible light.

11. A method as claimed in claim 1, wherein the at least one tethered reagent is adsorbed on to the transducer.

12. A method as claimed in claim 1, wherein the label is a gold particle having a particle size of 50-250 nm.

13. A method as claimed in claim 1, wherein the electromagnetic radiation has wavelength of 610 nm or above.

14. A method as claimed in claim 1, wherein the electromagnetic radiation has wavelength of 654 nm.

15. A kit comprising
(i) a device for detecting an analyte in a liquid sample containing suspended particles comprising
a radiation source adapted to generate a series of pulses of electromagnetic radiation at a wavelength of 600 nm or above,
a transducer having a pyroelectric or piezoelectric element and electrodes which is capable of transducing a change in energy to an electrical signal,
at least one tethered reagent on or proximal to the transducer, the tethered reagent having a binding site which is capable of binding the analyte, and
a confinement structure for holding the sample in fluid contact with transducer,
a detector which is capable of detecting the electrical signal generated by the transducer and which is adapted to determine the time delay between each pulse of electromagnetic radiation from the radiation source and the generation of the electric signal; and
(ii) a labelled reagent which has a binding site which binds to the analyte or the tethered reagent and a label which is capable of absorbing electromagnetic radiation generated by a radiation source to generate energy by non-radiative decay, wherein the label on the labelled reagent is selected such that the label absorbs the electromagnetic radiation at a level which is at least equal to the absorption of the sample of whole blood at the wavelength of the electromagnetic radiation used.

16. A kit as claimed in claim 15, wherein the at least one tethered reagent is an antibody and the analyte is an antigen.

17. A kit as claimed in claim 15, wherein the labelled reagent is a labelled antibody.

18. A kit as claimed in claim 15, wherein the at least one tethered reagent is an antibody, the analyte is an antigen and the labelled reagent is a labelled antigen which is also capable of binding to the at least one tethered reagent and the electrical signal detected by the detector is inversely proportional to the presence of the analyte in the sample.

19. A kit as claimed in claim 15, wherein the at least one tethered reagent is a first nucleic acid and the analyte is a second nucleic acid and the first and second nucleic acids are complementary.

20. A kit as claimed in claim 15, wherein the at least one tethered reagent contains avidin, streptavidin or derivatives thereof and the analyte contains biotin or derivatives thereof, or vice versa.

21. A kit as claimed in claim 15, wherein the label on the labelled reagent is selected from a dye molecule, a gold particle, a coloured-polymer particle, a fluorescent molecule, an enzyme, a magnetic particle, a carbon particle and a nanoparticle comprising a non-conducting core material and at least one metal shell layer.

22. A kit as claimed in claim 15, wherein the time delay is at least 20 milliseconds.

23. A kit as claimed in claim 15, wherein the time delay is no greater than 500 milliseconds.

24. A kit as claimed in claim 15, wherein the electromagnetic radiation is light, preferably visible light.

25. A kit as claimed in claim 15, wherein the at least one tethered reagent is adsorbed on to the transducer.

26. A kit as claimed in claim 15, wherein the confinement structure is a well.

27. A kit as claimed in claim 15, wherein the device is a lateral flow device and the confinement structure is a porous material.

28. A kit as claimed in claim 15, wherein the electromagnetic radiation has wavelength of 610 nm or above.

29. A method as claimed in claim 15, wherein the electromagnetic radiation has wavelength of 654 nm.
